(19) United States
(12) Patent Application Publication
(10) Pub. No.: US 2012/0263696 A1
(43) Pub. Date: Oct. 18, 2012

(54) Indirect Substrates for Microorganisms Metabolizing 1,2-Propanediol

(76) Inventor: Stefan ROOS, Uppsala (SE)

(21) Appl. No.: 13/400,735

(22) Filed: Feb. 21, 2012

Related U.S. Application Data
(60) Provisional application No. 61/517,130, filed on Apr. 14, 2011.

(51) Int. Cl. A61K 35/74; A61K 31/732; A61K 31/7004

(52) U.S. Cl. 424/93.45; 514/23; 514/54; 424/93.4

(57) ABSTRACT

The present invention relates generally to enhanced activity of certain probiotics. The increased efficacy is achieved by using certain substrate components that indirectly supply the probiotics with a specific source of energy. The substrate components are specifically designed to stimulate 1,2-propanediol production.

### CROSS-REFERENCE TO RELATED APPLICATIONS

This application claims priority from U.S. provisional applications Ser. No. 61/517,130, filed Apr. 14, 2011, and Ser. No. 61/463,846, filed Feb. 23, 2011.

### FIELD OF THE INVENTION

The present invention relates generally to enhancing the activity of certain probiotics in mammals. Moreover this invention relates to preparations comprising substrate components and certain probiotics, the substrate components being specifically designed to enhance the efficacy of said probiotics. The substrate components are selected to generate 1,2-propanediol, which uniquely most Lactobacillus reuteri strains can utilize as a source of energy and/or as an external electron acceptor.

### BACKGROUND OF THE INVENTION

This invention discloses a method of enhancing the activity of certain probiotics and the manufacturing and use of products, which comprises substrate components and optionally a probiotic. The products of the present invention can be used to enhance the activity of for example L. reuteri in mammals. This product can be used for improving the host health. Depending on the probiotic strain that is used, the product can be used for example to improve gastrointestinal health, improve immune-related health, treat and/or prevent diarrhea and constipation, normalize fecal consistency, improve gastrointestinal motility, treat and/or prevent infectious diseases, modulate inflammation and anti-pathogenic effect.

The Food and Agricultural Organization of the United Nations define probiotics as “live microorganisms which when administered in adequate amounts confer a health benefit on the host”. Nowadays, a number of different bacteria are used as probiotics for example, lactic-acid producing bacteria such as strains of Lactobacillus and Bifidobacterium.

The effectiveness of probiotics is strain-specific, and each strain may contribute to host health through different mechanisms. Different probiotics can prevent or inhibit the proliferation of pathogens, suppress production of virulence factors by pathogens, modulate the immune response in a pro-inflammatory or an anti-inflammatory way and influence the host in a number of other ways.

Prebiotics are defined as “non-digestible food ingredients that beneficially affect the host by selectively stimulating the growth and/or activity of one, or a limited number of bacteria in the colon that can improve the host health.” Targets for prebiotics are usually bifidobacteria and lactobacilli: however, prebiotics are often not selective, and hence stimulation of beneficial genera or probiotic strains alone may be difficult to achieve. Since it is difficult to find a prebiotic that is selective for certain probiotics, the inventor of the present invention has discovered how to use specific substrate components (SSC) that indirectly will supply specific probiotics with a source of energy and/or an external electron acceptor that will increase the energy yield.

Lactobacillus reuteri is a heterofermentative lactic acid bacterium and is frequently found in the gastrointestinal tract of humans and other animals. L. reuteri is considered an indigenous organism of the human gastrointestinal tract and is for example present on the mucosa of the gastric corpus, gastric antrum, duodenum, and ileum. See, for example U.S. Pat. Nos. 5,439,678, 5,458,875, 5,534,253, 5,837,238, and 5,849,289. When L. reuteri cells are grown under anaerobic conditions in the presence of glycerol, they produce the antimicrobial substance known as reuterin (B-hydroxy propionaldehyde). The ability to produce reuterin is due to the propanediol utilization (pdu) machinery, which is made possible by microorganisms having a pdu-operon. The pdu-machinery (pdu-operon) is a metabolic machinery that also enables growth on 1,2-propanediol (PD). This pdu-machinery is a very important feature for L. reuteri when colonizing humans and exploiting the full potential of the bacteria. This machinery is rare among other lactobacilli and therefore those without the pdu-machinery are not able to grow on 1,2-PD and neither are they capable of using 1,2-PD as an electron acceptor.

Different L. reuteri strains have different ability to colonize the intestine, act as a diarrhea therapeutic agent, modulate the gut motility, function as an inhibitor of bacterial pathogens, immunologically modulate the gastrointestinal mucosa, function as an anti-inflammatory agent in the stomach etc.

In patent application WO2009/151391, the pdu machinery of L. reuteri is primed with 1,2-PD or glycerol before freeze-drying the bacteria. With this manufacture design the freeze-dried pdu machinery of L. reuteri is primed with the capacity to make and store reuterin.

Emma Arsköld et. al., Phosphoketolase Pathway Dominates in Lactobacillus reuteri ACC 55730 Containing Dual Pathways for Glycolysis (Journal of Bacteriology, January 2008, p. 206-212) describes that growth performance of glucose can be improved by adding fructose as an external electron acceptor. However nothing in this article teaches how to select certain SSCs based on their ability to indirectly supply certain probiotics with 1,2-PD, which may only be utilized by bacteria with the pdu-machinery.

A common problem with oral administration of probiotic bacteria is insufficient amounts and/or activity of the probiotic bacteria in locations of the intestinal tract where they will assert their effects. This may have as a consequence that the dosage of probiotic bacteria has to be increased and/or more frequent administration is needed and might also result in loss of activity. This leads to unnecessary costs, undesirable frequency of intake and/or decreased health benefits. In the present invention the local amounts and/or metabolic activity of for example L. reuteri is enhanced, leading for example to the possibility of lowering the dosage of the probiotic and further that site-directed health benefits are possible.

1,2-propanediol (1,2-PD) is a source of energy that can be locally produced by other coexisting microorganisms and utilized, possibly in combination with additional sugars, by certain probiotic species, e.g. L. reuteri. The inventor of the present invention has surprisingly discovered that those coexisting microbes can be stimulated to produce 1,2-PD by oral administration of very selective SSCs and thereby indirectly enhance the activity of 1,2-PD utilizing organisms such as L. reuteri.

Pectin is a polysaccharide from plant cell walls. Various pectic polysaccharides can be detected in the cell wall, including homogalacturonan (HG), xylogalacturonan (XGA), apiogalacturonan, rhamnogalacturonan 1 (RGI), and rhamnogalacluronan U (RGII). The ratio between HG, XGA, RGI and RGII is variable, but typically HG is the most abundant polysaccharide constituting about 65% of the pectin, while RGI constitutes 20% to 35%. XGA and RGII are minor components, each constituting less than 10%. The different pectic polysaccharides are not separate molecules but covalently linked domains. L-rhamnose is found as a constituent in the pectin structures RGI and RGII. L-fucose is also found as a constituent in the RGII structure. Bacteria found in the GI-tract that are able to convert L-rhamnose or L-fucose belong for example to Bacteroides and Enterobacteria genera, including E. coli bacteria.

Pectin is resistant to human digestion, but is degraded to sugars and then further metabolized, for example to 1,2-propanediol, by bacteria in the small intestine and colon. Pectin stimulates bacterial growth in the small intestine and in the colon. Pectin is used as a remedy for diarrhea, is related to improved intestinal environment and is also known to have anti-cancer properties. Modified citrus pectin (MCP) is citrus pectin that has been degraded to less complex molecules and is used to support cell growth and proliferation.

Fucoidan is a sulfated polysaccharide, with two distinct forms; F-fucoidan and U-fucoidan. F-fucoidan is to more than 95% composed of sulfated esters of fucose and U-fucoidan is approximately 20% glucuronic acid.

Galacto-oligosaccharides (GOS) generally comprise a chain of galactose units that arise through consecutive transgalactosylation reactions, with a terminal glucose unit, is classified as a prebiotic.

Lynch M B et al., The effect of dietary Laminaria-derived laminarin and fucoidan on nutrient digestablility, nitrogen utilisrttion, intestinal microflora and volatile fatty acid concentration in pigs (J Sci Food Agric. 2010 February; 90(3):430-7) have seen that pigs offered diets containing fucoidan have increased Lactobacillus spp. in the proximal colon and distal colon compared with non-fucoidan diets. Thus it is suggested that fucoidan may provide a dietary means to improve gut health in pigs. The increased Lactobacillus populations in feces due to a fucoidan diet have also been seen by J. V. O'Doherty et. al., The effect of dietary laminarin and fucoidan diet of the weanling piglet on performance and selected faecal microbial populations (Livestock science 2010 September).

However it was not previously known to select, for example, pectin and fucoidan, or fractions thereof, based on the amounts of L-rhamnose and/or L-fucose in order to generate 1,2-PD through bacterial fermentation and to use such compositions with high deoxy sugar content, particularly high L-rhamnose and/or L-fucose content, which will lead to high amounts of 1,2-PD thus indirectly supplying certain microorganism, for example L. reuteri with a source of energy and/or an external electron acceptor, which most other microbes are not able to utilize due to lack of the pdu machinery.

Patent application WO10117274 relates to a carbohydrate which is able to induce a detectable increase of a C5 and/or a C6 Short Chain Fatty Acid (SCFA). The SCFA has a positive effect on the gastrointestinal health of the subject treated. The carbohydrate used comprises pectin. Even though these workers chose pectins that may comprise traces of rhamnose, they did not disclose how to select and use specific pectin high in L-rhamnose or L-fucose to indirectly supply probiotics with the pdu-machinery with a specific source of energy and/or an external electron acceptor thus enhancing their activity.

U.S. Pat. No. 7,101,565 relates to a composition comprising a prebiotic and a probiotic. The prebiotic may comprise a pectin or pectic polysaccharide. However it is not disclosed in this invention how to select certain pectins, or use combinations with pectin and L-rhamnose or L-fucose, that will generate high amounts of 1,2-PD in the gastrointestinal tract, beneficial for probiotics with the pdu machinery.

In U.S. Pat. No. 5,902,578 an invention is disclosed that relates to a method of . preventing diarrhea associated with infectious agents such as rotavirus, or diarrhea associated with antibiotic therapies by using Lactobacillus. However in this invention Lactobacillus is not associated with additional SSCs for better efficacy.

Nobody has hitherto disclosed how to enhance the health promoting effects of certain probiotics by administering SSCs, together with a probiotic, e.g. L. reuteri, to indirectly supply such probiotics with a unique source of energy and/or an external electron acceptor. Oral administration of SSCs, with high content of L-rhamnose and/or L-fucose will secure the supply of 1,2-propanediol and thus indirectly supply certain probiotics with a source of energy and/or an external electron acceptor. This will increase the local amounts of health promoting microorganisms, e.g. L. reuteri and lead to better efficacy, making site directed effects possible.

Even though it has previously been known to use, for example, pectin together with probiotics, it was not previously known how to select SSCs based on their ability to form 1,2-PD for the indirect supply of certain probiotics with a specific energy source and/or a specific external electron acceptor.

Other objects and advantages will be more fully apparent from the following disclosure.

### SUMMARY OF THE INVENTION

The invention herein provides a method and products for enhancing the activity of certain probiotics. The increased efficacy of probiotics can be achieved by stimulating co-existing microbes to produce 1,2-propanediol (1,2-PD). The coexisting microbes are stimulated with certain specific substrate components (SSC) as described herein. The SSCs will ensure the presence of 1,2-PD in the gastrointestinal tract and indirectly supply certain beneficial organisms with 1,2-PD.

The ability to utilize 1,2-PD either as an energy source and/or as an external electron acceptor is unique for bacteria with the pdu-machinery and therefore the administration of SSCs will enhance the activity only of certain probiotics.

The SSCs can be administered together with the probiotics for enhancing the activity of the co-administered probiotics. The SSCs could also be administered alone, for example to increase the activity of previously administered probiotics.

1,2-PD, administered alone or generated by the SSCs, may further be combined with galacto-oligosaccharides (GOS) or other galactose containing saccharides to give an even better source of energy for the microorganisms.

Heterofermentative lactic acid bacteria produce lactate, ethanol and carbon dioxide using the phosphoketolase pathway (PKP). The PKP has poor energy yield compared to the Embden-Meyerhof pathway (EMP). This disadvantage can be compensated for by addition of external electron acceptors.

The inventor has surprisingly found out that by ensuring the presence of 1,2-propanediol in the gastrointestinal tract, the probiotics will simultaneously be supplied with a suitable external electron acceptor and thereby enhance the activity of the probiotic.

The SSCs of the present invention will selectively increase the growth of heterofennentative lactic acid bacteria, such as Lactobacillus reuteri, since they will provide the bacteria with a suitable electron acceptor enabling an enhanced activity.

L. reuteri are dependent on a good electron acceptor for growth in certain environments, the inventor of the present invention has surprisingly found out that 1,2-propanediol will serve as a good electron acceptor and can be supplied by the administration of SSCs.

Other objects and advantages of the present invention will become obvious to the reader and it is intended that these objects and advantages are within the scope of the present invention.

### BRIEF DESCRIPTION OF THE DRAWINGS

FIG. 1 is a graph showing growth of L. reuteri DSM 17938 in modified MRS (with no glucose and citrate) with addition of 1,2-PD, galactose and a combination thereof.

FIG. 2 is a table showing the increase of fecal amounts of L. reuteri with patients where L. reuteri is administered together with SSCs compared to patients where L. reuteri is administered alone. Group A receive the powder sachets of example 1, and group B receive the same L. reuteri strain but no SSCs. The values are given as the average log₁₀ CFU per gram of feces ±SEM.

### DETAILED DESCRIPTION OF THE INVENTION AND PREFERRED EMBODIMENTS THEREOF

The propanediol utilization (pdu) machinery of certain probiotics, among them for example Lactobacillus reuteri, enables growth on 1,2-propanediol (1,2-PD) as energy source and enables the ability to use 1,2-PD as an external electron acceptor.

In summary, and as discussed in more detail below, the invention herein is a method for enhancing the activity or increasing the growth of probiotic bacteria having a pdu-operon, in the gastrointestinal tract of an individual comprising administering a substance having the capacity to be metabolized to 1,2-propanediol in the gastrointestinal tract of the individual, and a composition comprising probiotic bacterial having a pdu-operon and a substance comprising a deoxy sugar that can be so metabolized. The substance comprises a deoxy sugar which has the capacity to be metabolized to 1,2-propanediol in the gastrointestinal tract of said individual, preferably is selected from the group consisting of rhamnose, fucose, pectin. rhamnose in combination with pectin, fucose in combination with pectin, and a combination of rhamnose, fucose and pectin, and most preferably is selected from the group consisting of rhamnose and fucose. The substance may be administered separately from administration of bacteria having a pdu operon or may be administered simultaneously with bacteria having a pdu operon. Preferably the bacteria having the pdu-operon comprise Lactobacilllus reuteri. Preferably the substance is administered to the individual as a daily dose range of 0.25-25 grams, and most preferably as a daily dose in the range of 1-2 grams. The method may further comprise simultaneously administering a saccharide comprising galactose, for example a galacto-oligosaccharide.

The inventor of the present invention has surprisingly found a way of enhancing the activity of certain probiotics, using certain specific substrate components (SSCs) to indirectly support a specific probiotic organism with 1,2-PD that may be utilized as a source of energy or as an external electron acceptor. Oral administration of these carefully selected SSCs stimulates coexisting microbes to produce 1,2-PD, which leads to locally produced 1,2-PD that can serve as a source of energy or as an external electron acceptor for certain microorganisms with the the pdu-machinery, such as for example L. reuteri. The SSCs may be administered alone or together with the probiotics.

The SSC is a substance. According to one embodiment of the invention, the substance is a substrate. In one embodiment, the substance consists of one component only. In another embodiment, the substance comprises two or more components.

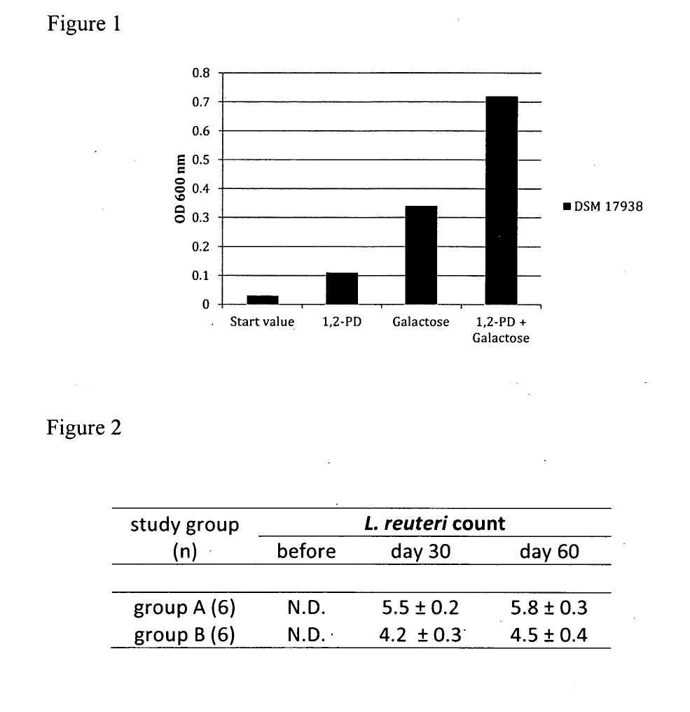

The presence of 1,2-PD improves the growth conditions for certain microorganisms. Moreover the inventor has found out that this can be further enhanced in the presence of GOS or galactose, as can be seen in FIG. 1.

The SSCs of the present invention are chosen for their ability to indirectly support a specific probiotic with 1,2-PD, which may be used as energy source or as an external electron acceptor. The inventor of this invention has discovered that SSCs with high amounts of L-rhamnose or L-fucose are the most effective when used to enhance the activity of certain probiotics. Therefore the SSCs used in this invention are carefully selected with regards to the amounts of L-rhamnose and L-fucose. The following examples of SSCs are not intended to limit the scope of this invention, but to exemplify the kind of components that could be used.

Pectins, preferably certain fractions of pectin comprising high amounts of rhamnogalacturonan I and II may be used as SSCs. These fractions of pectin will when degraded result in more rhamnose than ordinary pectin and thus generate higher amounts of 1,2-PD, preferably these fractions of pectin comprises 5-15% rhamnose. Ordinary pectin may also be used if administered together with L-rhamnose or L-fucose to indirectly and in the same manner be advantageous for certain microbes, e.g. L. reuteri. Further, the inventor of the present invention has shown that 1,2-PD in combination with certain pectin constituents, e.g. galactose, arabinose and galacturonic acid generates a synergistic effect that enhances the utilization of 1,2-PD as seen in FIG. 1.

Fucoidan, preferably certain fractions of fucoidan comprising high amounts of L-fucose may be used as SSCs in the present invention. These fractions of fucoidan when degraded result in more fucose than ordinary pectin and thus generate higher amounts of 1,2-PD.

L-rhamnose or L-fucose alone may also be used as SSCs of this invention.

It is also a possibility to combine L-rhamnose or L-fucose with ordinary pectin. This combination, in addition to 1,2-PD, also supplies certain probiotics with other substrates, e.g. galactose, arabinose and galacturonic acid as a result of pectin degradation. This combination of substrates has been shown to enhance the utilization of 1,2-PD for certain probiotics, e.g. L. reuteri.

Other SSCs, e.g. gums and other polysaccharides, can be used according to the present invention if they contain L-rhamnose, L-Fucose or the like. Gums include, but are not limited to karaya gum and arabic gum. Further, some HMO's from human breast milk can be used as SSCs in the present invention.

Administering the SSCs of the present invention, either alone or together with a probiotic, e.g. L. reuteri, secures the supply of energy source for a specific probiotic and/or secures the presence of an external electron acceptor that increases the energy yield, thus enhancing the local activity and efficacy of said probiotic.

In other embodiments and to support this effect it is further possible to add GOS or galactose in order to increase L. reuteri's ability to utilize 1,2-PD.

Depending on the target indication, a number of L. reuteri strains can be used in the invention herein with different ability to colonize the intestine, act as a diarrhea therapeutic agent, modulate the gut motility, function as an inhibitor of bacterial pathogens, immunologically modulate the gastrointestinal mucosa, function as an anti-inflammatory agent in the stomach etc.

Pectin, L-rhamnose and L-fucose are resistant to human digestion, but are degraded to sugars and then further metabolized to for example 1,2-propanediol by co-existing microbes found in humans and available at certain locations of the human GI-tract, for example on the mucosa of the gastric corpus, gastric antrum, duodenum, and small intestine. The invention herein therefore also makes it possible to favor site-directed effects in the human GI-tract. For example, by using selected strains of L. reuteri as the probiotic, it is possible to enhance the anti-inflammatory effect of this strain in the ileum.

When using the described system of certain SSC's in the present invention, with for example a specific strain of L. reuteri in humans with a very disturbed microflora, including a lack of normally found co-existing microbes able to convert the SSC to 1,2-PD, it is also another possibility of the invention herein to actively supply such co-existing microorganisms, e.g. Lactobacillus rhamnosus, to the recipient ensuring the efficacy of the administered SSCs.

The products comprising the SSCs of this invention, alone or in combination with certain probiotics are preferably, but not limited to, formulated as a tablet, capsule, powder sachet or the like. The product can be a food-supplement, a pharmaceutical product or the like. In such product the amount of probiotic feed should be in a sufficient amount to give the wanted effect of the specific strain, now also considering the enhanced effect by the SSC. Such levels are typically, but not limited to 10E+4 CFU to 10E+11 CFU per day, preferably in the range of 10E+6 CFU to 10E+9 CFU of L. reuteri.

The amount of the SSC should be in the range of 0.25 to 25 g per day when using pectins with high amounts of L-rhamnose and/or L-fucose and when using a combination of regular pectin with separate L-rhamnose and/or L-fucose, the total amount of SSCs should be in the range of 0.25 to 25 g per day. Even more preferably SSCs are administered in the range of 1-2 gram per day. The ratio between the regular pectin and either one of L-rhamnose or L-fucose, or the combination thereof, should be in the range of 95:5 to 0:100, but preferably 80:20 to 20:80 and even more preferably 70:30.

Another option when using the invention herein is to alternatively feed the SSC and the probiotic together, and at one or more occasions following the first feeding to feed only the SSC in a kind of shuttle program to lower the cost of treatment.

It is essential for this invention that the probiotics used have the pdu-machinery, since this is essential for the ability to use 1,2-PD as a source of energy and/or as an external electron acceptor. Therefore in another embodiment of the invention the pdu machinery of the probiotics is primed with 1,2-PD during the production of the probiotic strain for enhanced efficacy. This is done by adding 1,2 PD or glycerol and possibly cobalt or vitamin B-12 (since vitamin B-12 and cobalt are important for reuterin production) at the start of the fermentation step when culturing the bacteria. With this manufacture design, the freeze-dried bacteria to be used in the next step are better prepared to more quickly activate the pdu-machinery. This enhanced efficacy of the pdu machinery will in turn enhance the efficacy of the administered SSC of the present invention. Another way of enhancing the efficacy of the administered SSCs is to combine them with GOS or galactose because the inventor has shown that the combination of 1,2-PD and galactose has an unexpected benefit on L. reuteri's growth.

Since numerous modifications and changes will readily occur to those skilled in the art, it is not desired to limit the invention to the exact construction and operation shown and described, and accordingly, all suitable modifications and equivalents may be resorted to, falling within the scope of the invention.

### Example 1

### Manufacture of a Sachet Containing a Composition of L. reuteri Together With Pectin, Rhamnose and galacto-oligosaccharides

The composition is made of:

L. reuteri DSM 17938: 10E+8 CFU/sachet

Pectin (GENU® pectin (citrus) type USP/200, CP Kelco France SARL, France): 840 mg/sachet

L-rhamnose: (Rhamnose monohydrate L-(+). Kaden Biochemicals GmbH, Hamburg, Germany) 360 mg/sachet

GOS15 (VIVINAL®, FrieslandCampina Domo, The Netherlands) 800 mg/sachet

The composition is filled at ambient temperature into aluminum foil bags as known in the art with desiccant (10 cm×12 cm, using packaging material PET12/PE/ALU 12/PE/PE+desiccant/PE from Alcan) in a LAF bench (Holten Laminair Model S-2010 1.2 from Heto-Holten A/S, Denmark). To each bag, 2 g of powder with L. reuteri, pectin, L-rhamnose and galacto-oligosaccharides is added using the balance XP-600 from Denver Instrument GmbH, Germany. The filled aluminum foil bags are then heat sealed with the film sealing device model F460/2 from Kettenbaum Folienschweisstechnik GmbH & Co. KG, Germany.

### Example 2

### Manufacture of a Sachet Containing a Composition of L. reuteri Together With Rhamnose

The composition is made of:

2 g L-rhamnose: (Rhamnose monohydrate L-(+), Kaden Biochemicals GmbH, Hamburg, Germany)) containing 10E+8 CFU L. reuteri DSM 17938/sachet

The composition is filled into aluminum foil bags as in Example 1.

### Example 3

### Manufacture of a Sachet Containing a Composition of L. reuteri Together With galacto-oligosaccharides and Rhamnose

The composition is made of:

1 g GOS15 (VIVINAL®, FrieslandCampina Domo, The Netherlands) and 1 g L-rhamnose. (Rhamnose monohydrate L-(+), Kaden Biochemicals GmbH, Hamburg, Germany) containing 101E+8 CFU L. reuteri DSM. 17938/sachet

The composition is filled into aluminum foil bags as in Example 1.

### Example 4

### Manufacture of a Primed L. reuteri Strain

This example describes how to manufacture a freeze-dried powder of L. reuteri with activated pdu-machinery. The primed L. reuteri strain can then be used when producing the sachets of example 1.

Fermentation Medium Composition

Dextrose mono hydrate 60 g/l

Yeast extract KAV 20 g/l

Peptone Type PS (of pig origin) 20 g/l

Di ammonium hydrogen citrate 5 g/l

Sodium acetate (x 3 H₂O) 4.7 g/l

Di potassium hydrogen phosphate 2 g/l

Tween80 0.5 g/l

Silibione (anti foam) 0.14 g/l

Magnesium sulphate 0.10 g/l

Manganese sulphate 0.03 g/l

Zinc sulphate hepta hydrate 0.01 g/l

Water q.s.

Centrifuge Medium

Peptone O-24 Orthana (of pig origin)

Cryoprotectants

Lactose (of bovine origin) 33%

Gelatin hydrolysate (of bovine origin) 22%

Sodium glutamate 22%

Maltodextrin 11%

Ascorbic acid 11%

Production Steps of Freeze Dried Lactobacillus reuteri Powder

1. Twenty ml of the fermentation medium is inoculated with 0.6 ml of freeze-dried Lactobacillus reuteri powder from a working cell bank vial. The fermentation is performed in a bottle at 37° C. for 18-20 hours without stirring or pH control i.e. static.

2. Two 1-liter flasks of the fermentation medium are inoculated with 9 ml cell slurry (from step 1) per liter of medium. The fermentation is performed at 37° C. for 20-22 hours without stirring or pH control i.e. static.

3. The two one liter cell slurries from step no. 2 are used to inoculate the 600-liter vessel containing 600 liters fermentation medium. The fermentation is performed at 37° C. for 13 hours with stirring and pH control. At the start of the fermentation the pH is 6.5. The pH control starts when the pH drops below 5.4 using a 20% sodium hydroxide solution. The pH control is set to pH 5.5.

4. The fourth and final fermentation step is performed in a 15,000-liter vessel with the inoculation from step no. 3. The fermentation is performed at 37° C. for 9 to 12 hours with stirring and pH control. At the start of the fermentation the pH is 6.5. The pH control starts when the pH drops below 5.4 using a 20% sodium hydroxide solution. The pH control is set to pH 5.5. 100 mM glycerol is added in the final phase of the fermentation, just before the culture reaches the stationary phase. The fermentation is complete when the culture reaches the stationary phase, which can be seen by the reduction of the addition of the sodium hydroxide solution. Roughly 930 liters of the sodium hydroxide solution is added to the 10,200 liters of media and 600 liters of inoculum during the fermentation.

The cell slurry from the final fermentation (step 4) is separated at 10° C. twice in a continuous centrifuge from Alfa Laval. By the first centrifugation the volume of the cell slurry is reduced from roughly 11,730 liters to 1200 liters. This volume is washed with 1200 liters of a peptone (Peptone 0-24, Orthana) solution in a 3000-liter vessel and is separated again before the mixing with the cryoprotectants (see below). The washing step with peptone is performed to avoid any freezing-point reduction in the freeze-drying process.

By the second centrifugation the volume of the cell slurry is reduced to 495 liters. This volume is mixed with 156 kg of the cryoprotectant solution to reach roughly 650 liters of the cell slurry.

The cell slurry is pumped to a 1000-liter vessel. The vessel is then transported to the freeze-drying plant.

At the freeze-drying plant, exactly 2 liters of the cell slurry is poured on each plate in the freeze dryer. The maximum capacity of the freeze dryer is 600 liters and all excessive cell slurry volume is thrown away.

The cell slurry of Lactobacillus reuteri has a dry matter content of 18% and is freeze dried for four to five days.

During the freeze-drying process, the pressure in the process is between 0.176 mbar and 0.42 mbar. The vacuum pump is started when the pressure reaches 0.42 mbar. The PRT (pressurizing test) is used to determine when the process is complete. If the PRT or the increase of pressure is less then 0.02 mbar after 120 seconds, the process is stopped.

### Example 5

### Combination of 1,2-PD and galactose Generates a Synergistic Effect Enhancing the Activity of L. reuteri

L. reuteri DSM 17938 was grown in modified MRS broth (with no glucose and citrate) with addition of 1,2-PD (0.3%), galactose (0.3%) or a combination of the two. The bacteria were grown for 24 h at 37° C. L. reuteri grown in the presence of both 1,2-PD and galactose surprisingly showed a significantly higher growth than the growth on the separate substances as seen in FIG. 1.

### Example 4

### Example of Shuttle Program to be Fed

Thanks to the enhanced activity of L. reuteri induced by the SSCs described in this application it is possible to alternate the sachets of example 1 (sachet A) with sachets where L. reuteri is excluded but otherwise manufactured according to example 1 (sachet B) in a shuttle program. This shuttle program does not reduce the efficiency of L. reuteri and may lower the treatment cost.

Sachet A is administered to the recipient at day 1, at day 2 and 3 the recipient is given sachet B. This administration scheme is repeated during the whole treatment period.

### Example 5

### Intestinal Colonization in Vivo in Humans

A comparison between the intestinal colonization by L. reuteri alone and the same L. reuteri administered together with SSC is made in a clinical study. 12 healthy volunteers are divided into two groups (A and B) with 6 participants in each group. Group A receive the powder sachets of example 1, group B receive the same L. reuteri strain but no SSCs. Both groups are given 10E+8 CFU of L. reuteri per day during 60 days. The quantitative evaluation of intestinal colonization by strains given alone or together with the SSCs is made by fecal sample examination at the beginning of the study, and after 30 and 60 days of the treatment period. Fecal L. reuteri is counted and the fecal amounts of group A and B are compared.

A significant increase in the fecal amounts of L. reuteri is seen with patient where L. reuteri is administered together with SSCs compared to patients where L. reuteri is administered alone, as seen in FIG. 2.

While the invention has been described with reference to specific embodiments, it will be appreciated that numerous variations, modifications, and embodiments are possible, and accordingly, all such variations, modifications, and embodiments are to be regarded as being within the spirit and scope of the invention.

What is claimed is:

1. A method for enhancing the activity of probiotic bacteria having a pdu-operon, in the gastrointestinal tract of an individual, comprising administering a substance to said individual, which substance has the capacity to be metabolized to 1,2-propanediol in the gastrointestinal tract of said individual.

2. The method of claim 1, wherein said substance comprises a deoxy sugar which has the capacity to be metabolized to 1,2-propanediol in the gastrointestinal tract of said individual.

3. The method of claim 2, wherein said deoxy sugar is selected from the group consisting of rhamnose and fucose.

4. The method of claim 1, wherein said substance is selected from the group consisting of rhamnose, fucose, pectin, rhamnose in combination with pectin, fucose in combination with pectin, and a combination of rhamnose, fucose and pectin.

5. The method of claim 1, wherein the substance is administered separately from administration of bacteria having a pdu operon.

6. The method of claim 1, wherein the substance is administered simultaneously with bacteria having a pdu operon.

7. The method of claim 1, wherein the bacteria having the pdu-operon.comprise Lactobacilllus reuteri.

8. The method of claim 1, wherein the substance is administered to the individual as a daily dose range of 0.25-25 grams.

9. The method of claim 8, wherein the substance is administered to the individual as a daily dose in the range of 1-2 grams.

10. The method of claim 1, further comprising simultaneously administering a saccharide comprising galactose.

11. The method of claim 10, wherein the saccharide is a galacto-oligosaccharide.

12. A composition comprising probiotic bacteria having a pdu-operon, and a substance that has the capacity to be metabolized to 1,2-propanediol in the gastrointestinal tract of said individual.

13. The composition of claim 12, wherein said substance comprises a deoxy sugar which has the capacity to be metabolized to 1,2-propanediol in the gastrointestinal tract of said individual.

14. The composition of claim 13, wherein said deoxy sugar is selected from the group consisting of rhamnose and fucose.

15. The composition of claim 12, further comprising a substance selected from the group consisting of rhamnose, fucose, pectin, rhamnose in combination with pectin, fucose in combination with pectin, and a combination of rhamnose, fucose and pectin.

15. The composition of claim 12, further comprising a saccharide comprising galactose.

16. The composition of claim 15, wherein the saccharide comprising galactose is a galacto-oligosaccharide.

17. The composition of claim 12, wherein the bacteria having the pdu operon comprise Lactobacillus reuteri.

18. The composition of claim 12, wherein the substance is present in an amount to give a daily dose of 0.25-25 grams.

19. The composition of claim 18, wherein the substance is present in an amount to give a daily dose of 1-2 grams.
